(19) United States
(12) Patent Application Publication
(10) Pub. No.: US 2022/0096373 A1
(43) Pub. Date: Mar. 31, 2022

(54) Bandage Contact Lens for Sustained Drug Delivery

(71) Applicant: The Regents of the University of California, Oakland, CA (US)

(72) Inventors: Albert Fan Yee, Irvine, CA (US); Xin Fu, Oakland, CA (US); Sara Elizabeth Heedy, San Clemente, CA (US)

(73) Assignee: The Regents of the University of California, Oakland, CA (US)

(21) Appl. No.: 17/403,709

(22) Filed: Aug. 16, 2021

Related U.S. Application Data
(60) Provisional application No. 63/065,981, filed on Aug. 14, 2020.

(51) Int. Cl. A61K 9/00; A61K 31/7048; A61K 31/506; A61K 9/51

(52) U.S. Cl. CPC A61K 9/0051; A61K 31/7048; A61K 9/5161; A61K 9/5115; A61K 31/506

(57) ABSTRACT

A drug-loaded, flexible and transparent composite material for use in bandages and bandage contact lenses to topically administer a medication in a sustained and effective way is described, as well as methods of fabrication and use thereof. The composite material comprises drug carrying porous nanoparticles embedded within a transparent, flexible polymeric matrix and, when applied as a bandage, provides topical drug delivery to a tissue, while protecting it from the environment, yet allowing facile examination due to transparency.

### CROSS-REFERENCE TO RELATED APPLICATIONS

The present application claims priority under 35 U.S.C. 119(e) to U.S. Provisional Application No. 63/065,981, entitled “Bandage Contact Lens For Sustained Drug Delivery” to Yee et al., filed Aug. 14, 2020, the disclosure of which is incorporated herein by reference in its entirety.

### STATEMENT REGARDING FEDERALLY SPONSORED RESEARCH

This invention was made with Government support under Grant No. W81XWH-17-1-0355 awarded by the ARMY. The Government has certain rights in this invention.

### FIELD OF THE INVENTION

The invention is generally directed to a transparent, flexible composite material for topical drug delivery, including for use in bandages, and, more specifically, to a bandage contact lens for ocular drug delivery for treatment or prophylaxis of ocular diseases, and to methods of fabrication and use thereof.

### BACKGROUND OF THE INVENTION

While eyes occupy only 0.1% of the total body surface, the impact of their dysfunction is tremendous due to the importance of vision. Examples of ocular afflictions include: glaucoma, inflammation, allergies, dry eye disease, microbial infections, and eye trauma in general. Furthermore, improper treatment of ophthalmic diseases may cause eye sensitivity, poor visual acuity, decreased life quality, and even loss of a career in the long term. Many of the ocular diseases are treated by administration of topical medications. However, topical medications offer low bioavailability, wherein, typically, only ˜2 to 5% of a medication reaches the target tissue. Furthermore, there is a high chance of side effects from topical medications, for example, from a burst release of a drug. In addition, the administration of topical medications often requires difficult to achieve high patient compliance, for example, frequent applications of eye drops by a patient, and, as a result, unstable therapeutic outcomes. Considering an estimation of 16 million cases of dry eye disease in US (see, for example, Farrand, K. F., et al. Am J Ophthalmol, 182: 90-98 (2017), the disclosure of which is incorporated herein by reference), 2.7 million cases of glaucoma in US (National Institute of Health, Projections for glaucoma (2010-2030-2050), https://www.nei.nih.gov/learn-about-eyehealth/resources-for-health-educators/eye-health-data-and-statistics/glaucoma-data-and-statistics/glaucoma-tables, retrieved May 13, 2020, the disclosure of which is incorporated herein by reference), more than 2 million cases of keratitis (microbial infections of cornea) globally (Ung, L., et al. Surv Ophthalmol. 64, 255-271 (2019), the disclosure of which is incorporated herein by reference), and the demand for a reliable treatment for eye injuries in general (including prophylactic antibiotic treatment), there exists a consistent need for effective and reliable methods of ocular drug application.

### SUMMARY OF THE INVENTION

Various embodiments are directed to a bandage contact lens for topical administration of ophthalmic drugs comprising
a composite material comprising:
   a matrix comprising a polymeric material;
   a plurality of porous nanoparticles embedded within the matrix and characterized by a mean particle diameter and a pore diameter; and
   at least one drug for treatment of an ophthalmic condition loaded into the plurality of porous nanoparticles; wherein
      the composite material is transparent and flexible, and delivers the at least drug to an eye topically and consistently upon contact over a period of time, while also protecting the eye from the environment.

In various such embodiments, the ophthalmic condition is a medical condition selected from the group consisting of: dry eye disease, eye allergy, glaucoma, microbial infection, inflammation, post-trauma and post-surgery care, any combination thereof, and any other ocular condition that can be treated by topical administrations of a drug.

In still various such embodiments, the at least one drug is selected from the group consisting of: antibiotics, antifungals, anti-inflammatories, antiglaucoma agents, antihistamines, eye lubricants, medications and supplements for dry eye disease, medication for any other ocular diseases treatable via topical administration, and any combination thereof.

In yet various such embodiments, the at least one drug is selected from the group consisting of: natamycin, voriconazole, and any combination thereof.

In still yet various such embodiments, the plurality of porous nanoparticles is a material selected from the group consisting of: mesoporous silica nanoparticles, dendritic mesoporous silica nanoparticles, zeolite nanoparticles, and any combination thereof.

In various such embodiments, the mean particle diameter is less than 400 nm.

In still various such embodiments, the mean particle diameter is less than 200 nm.

In yet various such embodiments, the plurality of porous nanoparticles is chemically modified at the surface.

In still yet various such embodiments, the matrix comprises an inherently antimicrobial material.

In various such embodiments, the matrix comprises a material selected from the group consisting of: a biopolymer, any commercial polymer used in manufacturing of soft contact lenses, including polyhydroxyethylmethacrylate, polydimethylsiloxane, polydimethylsiloxane copolymers, and any combination thereof.

In still various such embodiments, the biopolymer is a polymer selected from the group consisting of: chitosan, chitosan crosslinked with genipin or tripolyphosphate, silk Fibron, gelatin, any combination thereof.

In yet various such embodiments, the period of time of drug release from the bandage contact lens is at least 8 hours.

In still yet various such embodiments, the plurality of porous nanoparticles is dendritic mesoporous silica nanoparticles, the at least one drug is natamycin, and the matrix comprises chitosan crosslinked with tripolyphosphate.

Various other embodiments are directed to a bandage for topical drug delivery comprising
a composite material comprising:
   a matrix comprising a polymeric material;
   a plurality of porous nanoparticles embedded within the matrix and characterized by a mean particle diameter and a pore diameter; and
   at least one drug for topical treatment of a medical condition loaded into the plurality of porous nanoparticles; wherein
      the composite material is transparent and flexible, and delivers the at least one drug to a tissue topically and consistently upon contact over a period of time, while also protecting the tissue from the environment.

In various such embodiments, the at least one drug is selected from the group consisting of: antibiotics, antifungals, anti-inflammatories, antihistamines, lubricants, medication for any disease or condition treatable via topical administration, and any combination thereof.

In still various such embodiments, the plurality of porous nanoparticles is a material selected from the group consisting of: mesoporous silica nanoparticles, dendritic mesoporous silica nanoparticles, zeolite nanoparticles, and any combination thereof.

In yet various such embodiments, the plurality of porous nanoparticles is chemically modified at the surface.

In yet still various such embodiment, the matrix comprises an inherently antimicrobial material.

In various such embodiments, the matrix comprises a material selected from the group consisting of: a biopolymer, poly(hydroxyalkylmethacrylate), poly(alkylmethacrylate), poly(bisphenol-A carbonate), poly(alkylsiloxane), copolymers of poly(alkylsiloxane), and any combination thereof.

In still various such embodiments, the biopolymer is a polymer selected from the group consisting of: chitosan, chitosan crosslinked with genipin or tripolyphosphate, silk Fibron, gelatin, and any combination thereof.

In yet various such embodiments, the period of time is at least 8 hours.

Various embodiments are directed to a method of sustained topical administration of an ophthalmic drug comprising:
providing a bandage contact lens for topical administration of ophthalmic drugs comprising
   a composite material comprising:
      a matrix comprising a polymeric material;
      a plurality of porous nanoparticles embedded within the matrix and characterized by a mean particle diameter and a pore diameter; and
      at least one drug for treatment of an ophthalmic condition loaded into the plurality of porous nanoparticles; wherein
   the composite material is transparent and delivers the at least one drug to an eye topically and consistently upon contact over a period of time, while also protecting the eye from the environment; and
      applying the bandage contact lens to the eye to treat the ophthalmic condition.

Yet various other embodiments are directed to a method of sustained topical administration of a medicament comprising:
providing a bandage for topical drug delivery comprising
   a composite material comprising:
      a matrix comprising a polymeric material;
      a plurality of porous nanoparticles embedded within the matrix and characterized by a mean particle diameter and a pore diameter;
      at least one drug for treatment of a medical condition loaded into the plurality of porous nanoparticles; wherein
   the composite material is transparent and delivers the at least one drug to a tissue topically and consistently upon contact over a period of time, while also protecting the tissue from the environment; and
      applying the bandage to the tissue to treat the medical condition.

Still various other embodiments are directed to a composite material comprising:
a matrix comprising a polymeric material;
a plurality of porous nanoparticles embedded within the matrix and characterized by a mean particle diameter and a pore diameter; and
at least one drug for treatment of a medical condition loaded into the plurality of porous nanoparticles; wherein
   the composite material is transparent and flexible, and delivers the at least one drug to a tissue topically and consistently upon contact over a period of time, while also protecting the tissue from the environment.

In various such embodiments, the plurality of porous nanoparticles is a material selected from the group consisting of: mesoporous silica nanoparticles, dendritic mesoporous silica nanoparticles, zeolite nanoparticles, and any combination thereof.

In still various such embodiments, the matrix comprises a material selected from the group consisting of: chitosan, chitosan crosslinked with genipin or tripolyphosphate, silk fibron, gelatin, and any combination thereof.

Additional embodiments and features are set forth in part in the description that follows, and in part will become apparent to those skilled in the art upon examination of the specification or may be learned by the practice of the disclosed subject matter. A further understanding of the nature and advantages of the present disclosure may be realized by reference to the remaining portions of the specification and the drawings, which forms a part of this disclosure.

### BRIEF DESCRIPTION OF THE DRAWINGS

These and other features and advantages of the present invention will be better understood by reference to the following detailed description when considered in conjunction with the accompanying data and figures, wherein:

FIG. 1A provides a transmission electron microscope (TEM) image of the pore structure of a mesoporous silica nanoparticle (MSN) with the overall particle diameter of 300±60 nm and pore size of 4.4 nm, while FIG. 1B provides a scanning electron microscope (SEM) image of dendritic mesoporous silica nanoparticles (dMSNs) with irregular pore openings, wherein the nanoparticle diameter ranges from 60 to 80 nm and the pore size ranges from 6 to 10 nm (scale bar 500 nm), in accordance with prior art.

FIG. 2 provides a schematic of a bandage contact lens (BCL) for controlled topical drug delivery to an eye, wherein the inset image illustrates porous MSNs contained within such BCL, in accordance with embodiments of the application.

FIG. 3 demonstrates transparency of the composite material comprising 9 wt % of MSNs with mean diameter of 750 nm embedded into a chitosan film, in accordance with embodiments of the application.

FIGS. 4A through 4C provide images and data to illustrate the transparency properties of the composite material comprising 13 wt % of dMSNs with the particle diameters ranging from 60 to 80 nm, embedded within a chitosan matrix crosslinked with tripolyphosphate (TPP), wherein FIG. 4A demonstrates the transparency of: 1) (left image) a 2 cm×2 cm, 57 μm-thick, dry film sample comprising the composite material comprising dMSNs that were pre-loaded with 30 wt % voriconazole (to provide a total of 1 mg of voriconazole) and chitosan crosslinked with 10 wt % TPP; and 2) (right image) a dry lens of similar thickness (55 μm) comprising the composite material comprising empty dMSNs and chitosan crosslinked with 10 wt % TPP; FIG. 4B further demonstrates the transparency of the composite material comprising 13 wt % dMSNs with particle diameters ranging from 60 to 80 nm, embedded within a chitosan matrix crosslinked with 10 wt % TPP, wherein: 1) (top) the composite material comprises voriconazole-loaded dMSNs, and the composite material is provided in a form of a dry film with a thickness of 57 μm; 2) (middle) the composite material comprises empty dMSNs, and the composite material is provided in a form of a film with the pre-hydration thickness of 57 μm, shown before (left) and after (right) hydration/swelling with a phosphate buffer solution (PBS) at pH=7.4 for 24 hours; and 3) (bottom) the composite material comprises empty dMSNs, and the composite material is provided in a form of a contact lens with the pre-hydration thickness of 55 μm, shown before (left) and after (right) hydration/swelling with a phosphate buffer solution (PBS) at pH=7.4 for 24 hours; and FIG. 4C shows transmittance data collected across the visible range for a hydrated lens comprising empty dMSNs and chitosan crosslinked with either 5 wt % (solid line) or 10 wt % (dashed line) of TPP, in accordance with embodiments of the application.

FIG. 5 compares the drug release profiles from MSNs with different pore sizes, wherein the top plot shows the drug release profile for the pore-expanded, 4 nm pores MSNs, while the bottom plot shows the same for the regular, 2 nm pores MSNs, in accordance with embodiments of the application.

FIG. 6 compares the drug release profiles from a film comprising the composite material and a film comprising a chitosan matrix only, wherein the composite material film comprises voriconazole-loaded (30 wt %), 60 to 80 nm dMSNs (dMSN-30VRZ) embedded within the chitosan matrix (13 wt % of dMSNs) crosslinked with 10 wt % TPP, and contains the total voriconazole load of 1 mg, and wherein the chitosan only film comprises voriconazole directly loaded into chitosan crosslinked with 10 wt % TPP, and also contains the total voriconazole load of 1 mg, in accordance with embodiments of the application.

FIG. 7 provides an example of a fabrication sequence to produce the drug delivering bandage, in accordance with embodiments of the application.

### DETAILED DISCLOSURE

Turning now to the schemes, images, and data, a transparent, flexible composite material for sustained topical drug delivery, as well as methods of fabrication thereof, and methods of use thereof in bandages and bandage contact lenses (BCLs) for treatment and or prophylaxis of medical conditions, are described in accordance with various embodiments. Many embodiments are directed to the use of the composite materials in bandage contact lenses for topical treatment and or prophylaxis of ophthalmic diseases, while other embodiments are directed to the use of the composite materials in bandages for other types of organs and tissues. In many embodiments, the composite material at least comprises a plurality of porous nanoparticles dispersed within a flexible polymeric matrix, wherein the porous nanoparticles serve as drug vehicles. In many embodiments, the selection criteria for the components of the composite materials carefully balance the composite materials' drug-loading capacity and drug-release profile against the composite material's transparency and flexibility. It will be understood that the embodiments of the invention described herein are not intended to be exhaustive or to limit the invention to precise forms disclosed. Rather, the embodiments selected for description have been chosen to enable one skilled in the art to practice the invention.

Many eye conditions, including very serious ones, are treated by topical application of medication. However, topical formulae, such as eye drops, usually have low bioavailability of 2 to 5% and, therefore, require frequent application, which, in turn, may lead to low patient compliance and poor medical outcomes. For example, fungal keratitis, a noxious disease that can cause vision loss, is difficult to treat because it requires frequent eye drop administration of an antifungal drug natamycin or voriconazole.

In contrast, drug delivery by means of a bandage or a contact lens increases the time that a drug is continuously retained on the ocular surface, and, therefore, may increase bioavailability of the drug up to 50 to 70% (as predicted, for example, by Gause, S. et al. in Mechanistic modeling of ophthalmic drug delivery to the anterior chamber by eye drops and contact lenses. Advances in colloid and interface science 233, 139-154 (2016), the disclosure of which is incorporated herein by reference). Accordingly, such higher bioavailability allows for lower dosage and application frequency and, in turn, may lead to better patient adherence and medical outcomes. Therefore, contact lenses represent an appealing option for topical ocular drug delivery.

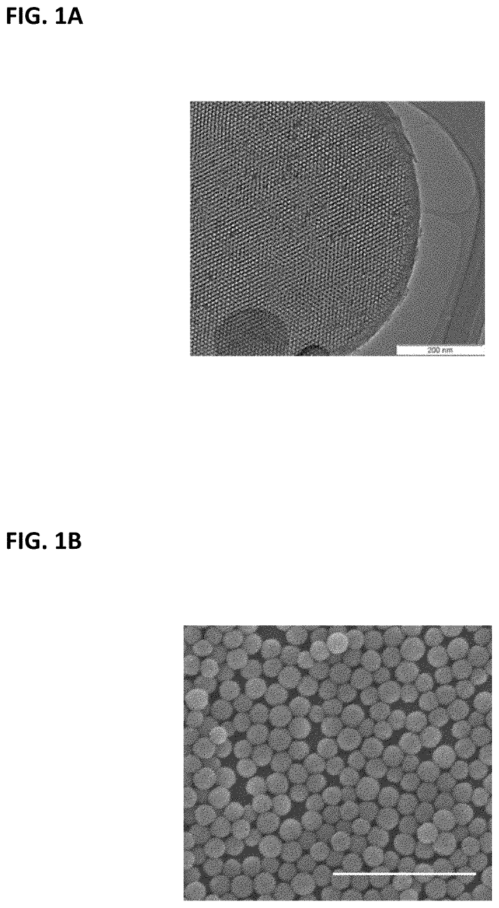

Mesoporous silica is a form of porous silica nanoparticles, illustrated, for example, in FIG. 1A (reproduced from Deodhar, G. V., et al. Langmuir 34, 228-233 (2018), the disclosure of which is incorporated herein by reference). To date, mesoporous silica nanoparticles (MSNs) have found applications in areas ranging from catalysis to medicine, biosensors, thermal energy storage and imaging. In particular, due to its many advantageous properties, mesoporous silica has recently gained attention as a material candidate for drug delivery applications. More specifically, the following aspects of mesoporous silica make it especially appealing for drug delivery applications: 1) silica is generally recognized as safe by the US Food and Drug Administration; 2) silica nanoparticles can be chemically modified in a variety of useful ways; and 3) the textural properties of MSNs, such as pore diameter, specific area, and overall particle size, can also be tuned. In turn, the control over surface chemistry and texture of nanoparticles in mesoporous silica materials allows for control over silica particle-drug interactions, including silica particle-drug electrostatic attraction, drug loading capacity of the particles, and drug release profile of the particles. For example, depending on the synthetic method used in MSN production, the size of particles in mesoporous silica materials can range from 30 nm to 1 micron, and even bigger, while the pore size of such particles can range from 1.6 nm to 50 nm. As a more specific example, the Stöber process, which utilizes self-assembly surfactants as pore templates, affords MSNs with evenly sized pores, with a narrow pore size distribution of within 1 nm, as described, for example, in Tang, F. et al. in Mesoporous silica nanoparticles: synthesis, biocompatibility and drug delivery. Advanced Materials, 24(12), 1504-1534 (2012), the disclosure of which is incorporated herein by reference. Accordingly, tuning both parameters—the overall particle size and the pore size—may increase specific area and loading capacity of MSNs, providing larger surface area for drug-silica interactions. As another example, designer chemical modifications of mesoporous silica particles can allow on-demand or triggered drug release, as well as minimization of drug loss during fabrication and storage of such drug delivery systems. Notably, incorporating, generally, as much as 10 wt % of MSNs (and not more than 15%) into a transparent polymer matrix does not significantly affect the transparency of the matrix, and results in a transparent composite material, as long as the particles' mean diameter is kept below 400 nm, and preferably below 200 nm.

However, MSNs with diameters of less than 200 nm are difficult to synthesize via conventional methods. Furthermore, smaller MSNs are expected to have smaller (evenly sized) pores/cavities, which, in turn, might limit the amount of a drug that can be loaded into such pores, wherein, for example, a drug molecule's size is large, and or, as another example, the molecular structure of a drug to be loaded is rigid. In contrast, dendritic mesoporous silica nanoparticles (dMSNs) fabricated via, for example, the procedure described by Huang et al. in Dendritic mesoporous silica nanospheres synthesized by a novel dual-templating micelle system for the preparation of functional nanomaterials. Langmuir, 33(2), 519-526 (2017), the disclosure of which is incorporated herein by reference, offer easy access to smaller nanoparticles that are characterized by a wider range distribution of pore diameters within a single silica nanoparticle. As such, the larger pores within dMSNs may grant easier entrance for drugs with large molecular dimensions without the need to increase the overall particle size to otherwise accommodate such drugs. For example, the overall diameter of dMSNs fabricated according to the Huang's methods (FIG. 1B) is less than 200 nm (i.e., 60 to 80 nm), while their pores' diameters range from 6 to 10 nm, as analyzed by Barrett-Joyner-Halenda (BJH) model with nitrogen adsorption assay. In other words, dMSN-based materials may allow for a broader scope of drugs that can be loaded into their smaller (as compared to MSNs) silica particles.

This application is directed to embodiments of a therapeutic and or prophylactic, transparent, flexible composite material for sustained topical drug delivery to a tissue. In particular, the application is directed to embodiments of a bandage contact lens (BCL), or another type of a transparent bandage, for topically delivering a medication to an eye, or another tissue, including to an open wound, in a sustained, effective, and reliable manner, to treat or prevent common ophthalmologic diseases, or other medical conditions requiring topical medication treatment. In many such embodiments, the composite material and, consequently, the BCL, or another bandage comprising the instant composite material, are transparent, yet provide a protective physical barrier against the environment, and are therapeutic and or prophylactic in a sustained manner over time. The transparency property is highly advantageous in a bandage because it allows for easy inspection of an underlying tissue, including by medical personnel, without having to remove the bandage. In the case of an ophthalmic device, the transparency of the bandage is critical, as it allows a patient to retain their vision, thus further increasing patient compliance.

Furthermore, in many embodiments, the composite material is flexible to allow for facile and comfortable application as a contact lens or a bandage. In many such embodiments, the composite material has a tensile modulus comparable to that of materials typically used in the manufacturing of soft contact lenses. In many embodiments, the tensile modulus of the composite materials falls into the 0.1 to 2.0 MPa range.

In many embodiments, the therapeutic and or prophylactic effect of the composite materials lasts a few hours, while in some embodiments the therapeutic and or prophylactic effect of the composite materials lasts a few days. In some embodiments, the therapeutic and or prophylactic effect of the composite materials lasts at least 8 hours. In some embodiments, the therapeutic and or prophylactic effect of the composite materials lasts at least 1 day.

Accordingly, in many embodiments, for example, a BCL comprising the composite material delivers a medication for treating an ophthalmic condition to an eye and protects the eye from further insult by microbial species, all while maintaining BCL's transparency. In many such embodiments, the BCL's transparency facilitates ophthalmic (or other) examination and enhances user experience. In many embodiments, the BCL can prevent and or treat ophthalmic afflictions including, but not limited to: dry eye disease, eye allergies, glaucoma, microbial infections, inflammations, post-trauma, post-surgery care, and other conditions and diseases that can be treated by topical administration of drugs. In some other embodiments, the composite material is applied to bandage another body part or tissue, that is not an eye, to protect and topically treat another, non-eye related, condition.

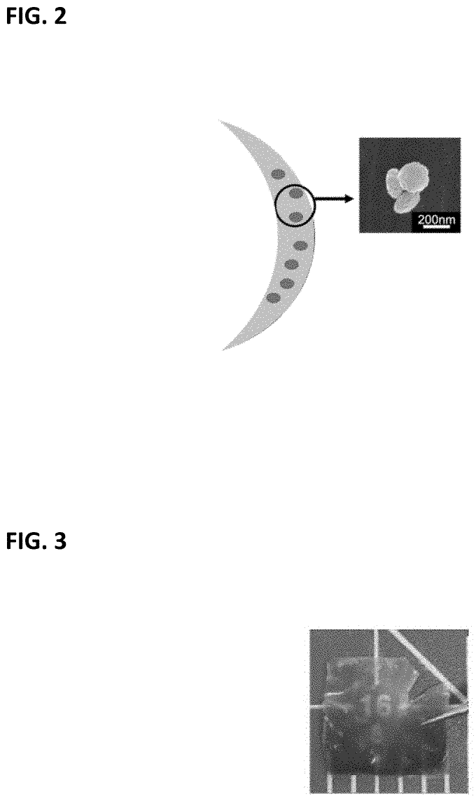

FIG. 2 provides a schematic of the bandage contact lens of many embodiments. In many such embodiments, the BCL, or another device or bandage, comprises the composite material of the instant disclosure, wherein the composite material further comprises a plurality of medicament-containing porous nanoparticles dispersed within a polymeric matrix. In many embodiments, the polymeric matrix comprises a polymer material used in the manufacturing of contact lenses. In many embodiments, the polymer matrix comprises any one or a combination of commercial polymers typically used in manufacturing of soft contact lenses, such as, for example, polyhydroxyethylmethacrylate (pHEMA), polydimethylsiloxane (PDMS, or silicone), PDMS copolymers, or a biopolymer, including, but not limited to chitosan, silk Fibron, or gelatin. However, in some embodiments, especially wherein the particular application does not critically require bandage softness and pliability, the polymer matrix may comprise one or more other transparent plastics, that might not be suitable for soft contact lenses, such as, for example, polycarbonates and polyalkylmethacrylates. Accordingly, the polymeric matrix of a non-BCL type bandage may comprise a material such as, but not limited to: a biopolymer, poly(hydroxyalkylmethacrylate), poly(alkylmethacrylate), poly(bisphenol-A carbonate), poly(alkylsiloxane), copolymers of poly(alkylsiloxane), and any combination thereof. In many embodiments, the material used as the polymer matrix is chosen to provide additional antimicrobial properties to the composite material of the disclosure. For example, chitosan is known to be inherently antimicrobial and, therefore, it is expected to endow the composite material and, correspondingly, the BCL, or another bandage, with additional antimicrobial properties. Accordingly, in some embodiments, the polymeric matrix comprises chitosan. In some embodiments, the polymeric matrix comprises chitosan hydrogel crosslinked with genipin or tripolyphosphate (TPP).

In many embodiments, the polymeric material of the polymeric matrix is additionally crosslinked for mechanical stability and or improved dispersion and or retention of the porous nanoparticles. In many such embodiments, the extent of the crosslinking of the polymeric matrix is carefully balanced against the overall stiffness and transparency of the resulting composite material. It should be noted that, the stiffness and transparency properties of the composite material are especially critical for BCL applications, because a contact lens that is too stiff or too opaque will be uncomfortable for its wearer. For example, a BCL comprising chitosan crosslinked with 10 wt % TPP may be mechanically stronger and more transparent than a similar BCL comprising chitosan crosslinked with only 5 wt % TPP, however, a BCL comprising chitosan crosslinked with more than 10 wt % TPP is expected to be too stiff for contact lens applications. Nevertheless, in some embodiments, wherein the composite material is used in drug delivering bandages for less sensitive tissues or organs than an eye, more extensive crosslinking of the polymeric matrix may be desired to optimize the bandage's robustness at expense of its softness and or transparency.

Moreover, in some embodiments, wherein, for example, the polymeric matrix comprises a material such as or similar to chitosan, the material's crosslinking density is used to affect the hydration/swelling ability of the matrix and, as such, the composite material's water content. For example, in many embodiments, higher crosslinking density results in lower water content in the matrix upon hydration/swelling. Not to be bound by any theory, water content is considered to be associated with oxygen permeability of conventional contact lens materials, wherein higher water content leads to higher oxygen permeability and greater user comfort, for example, by avoiding hypoxemia. Accordingly, in many embodiments, especially wherein the composite materials are used in BCL-type applications, the crosslinking of the matrix is minimized to allow for more hydrated and, therefore, more comfortable contact lenses.

Furthermore, embedding the porous nanoparticles into the polymeric matrix of the instant composite material also affects both the composite material's overall stiffness and transparency. For example, higher loading of the porous nanoparticles is expected to result in a stiffer and less transparent material, due to increased material density and particle clustering, which, in turn, causes more light to scatter. Accordingly, in many embodiments, especially wherein the composite material is used in BCL applications, the composite material's porous nanoparticle content is carefully balanced to ensure sufficient drug loading without sacrificing the resulting material's transparency and flexibility. However, in many embodiments, wherein the applications are less sensitive to transparency and stiffness parameters, such as, for example, non-eye wound dressing, the porous nanoparticle content may be varied over a broaden range to, for example, optimize for greater drug capacity. Overall, in many embodiments, both the polymeric matrix crosslinking density and the porous nanoparticle content are used as material optimization parameters to ensure that the composite material's stiffness and transparency fall within a suitable range for the end use application.

In many embodiments, the porous nanoparticles embedded into the polymeric matrix are nanoparticles selected from the group consisting of: mesoporous silica nanoparticles (MSNs), dendritic mesoporous silica nanoparticles (dMSNs), zeolite nanoparticles, and any combination thereof. In some embodiments, the porous nanoparticles are MSNs fabricated according to the protocol described in Deodhar et al., with or without modifications. In many embodiments, especially wherein a broader drug scope needs to be accommodated by a single composite material of the instant disclosure without sacrificing the composite material's transparency, dMSNs fabricated according to the protocol described in Huang, with or without modifications, are selected as the porous nanoparticles.

In many embodiments, the porous nanoparticles are selected for their particle size. In many embodiments, the size selection is dictated by competing material requirements, wherein, on one hand, bigger particles may be preferred for higher drug carrying capacity, while, on the other hand, smaller particles afford better overall material transparency. In many embodiments, similar considerations are also applied to nanoparticle concentration within the matrix, wherein a higher concentration of porous nanoparticle offers higher overall drug loading, but also decreases the composite material's transparency. As a more specific example, MSNs with the mean particle diameter of 400 nm have a lower matrix concentration limit for maintaining the transparency of the composite material than MSNs with the mean particle diameter of 200 nm. However, the 400 nm MSNs can accommodate a higher volume of drugs with faster drug release than the 200 nm MSNs. Accordingly, in many embodiments, the selected porous nanoparticles have the mean diameter of less than 400 nm to ensure composite material's transparency. In some embodiments, the mean diameter of porous nanoparticles is less than 200 nm.

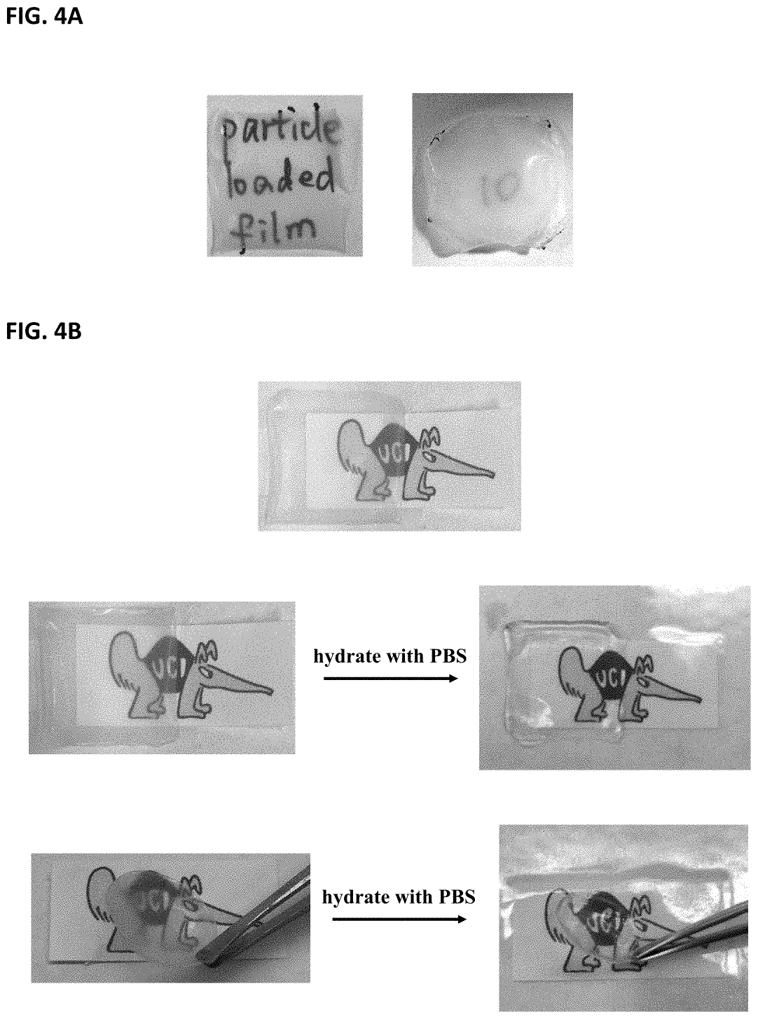

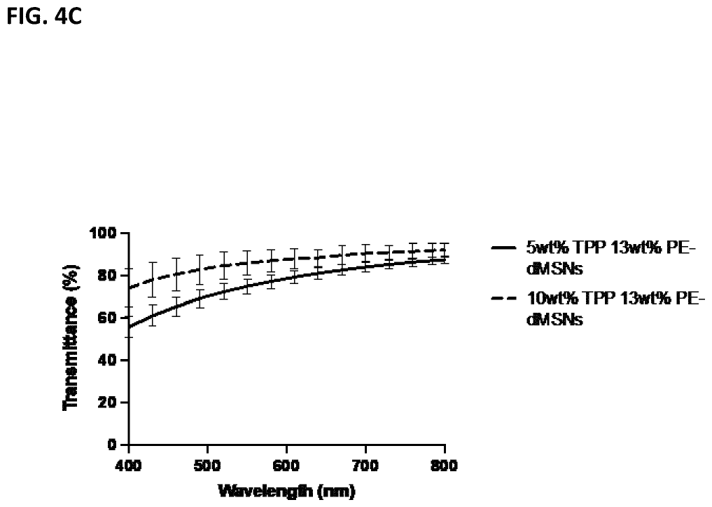

As another example, FIG. 3 illustrates the transparency of the composite material prepared according to the instant disclosure from MSNs with the mean diameter of 750 nm embedded into a chitosan film (9 wt % of empty MSNs). As seen from FIG. 3, 750 nm particles at such concentration still allow for some transparency, but the resulting transparency is not optimal. In contrast, the images in FIGS. 4A (left) and 4B (top and middle) demonstrate that a similar film comprising the composite material with a similar particle concentration (13 wt %) but much smaller particle size (60-80 nm) characteristic of dMSNs, affords much improved transparency. Furthermore, a concaved lens comprising the same dMSN-containing composite material also affords excellent transparency, as illustrated by the images in FIGS. 4A (right) and 4B (bottom). Notably, hydrating and swelling the composite material, with, for example, a phosphate buffer solution with pH=7.4, further increases the composite material's transparency as illustrated by FIG. 4B (middle and bottom). Not to be bound by any theory, the hydration is believed to reduce the refractive index mismatch between the composite material and its media and, therefore, leads to a higher material transmittance, as compared to a dry sample (FIG. 4B, middle and bottom).

FIG. 4C further illustrates the transparency properties of the composite materials of many embodiments comprising the porous nanoparticles with diameters of less than 200 nm, such as those afforded by dMSNs. More specifically, FIG. 4C provides transmittance data collected over the 400-800 nm range (i.e., visible range) for a hydrated lens comprising the composite material, wherein the composite material, in turn, comprises 13 wt % of 60 to 80 nm, empty dMSNs embedded within a chitosan matrix crosslinked with either 5 wt % (solid line) or 10 wt % (dash line) of TPP. As such, the lens wherein the matrix is crosslinked with 5 wt % TPP shows an average transmittance of 76%, while the lens wherein the matrix is crosslinked with 10 wt % TPP shows an average transmittance of over 86%, across the entire visible wavelength range. For comparison, Lira, M., et al. in Changes in UV-visible transmittance of silicone-hydrogel contact lenses induced by wear. Optometry and Vision Science, 86(4), 332-339 (2009), the disclosure of which is incorporated herein by reference, report a transmittance of above 79.12% at 400 nm, an average transmittance of above 83.90% at 400-700 nm for a typical commercial contact lens.

Notably, the data deviations (indicated by the error bars) for apparently identical samples observed in FIG. 4C may indicate clustering of the silica particles during the composite material fabrication process. Furthermore, the smaller scattering effect at shorter wavelengths may reflect a modulation of the optical interface between the silica particles and the surrounding matrix. Accordingly, in some embodiments, the surface of the porous nanoparticles is chemically modified to, for example, prevent nanoparticle clustering, or as needed in any other way, or for any other purpose. In many such embodiments, the chemical modification of porous nanoparticles adds surface functional groups or polymeric grafting that minimizes nanoparticle aggregation and ensures their adequate dispersion within the polymer matrix. For example, chemical modification or polymeric layer coating of the porous nanoparticles may be used to maintain charges on particles' surfaces to assist better suspension in solution during gel fabrication, and or to get a more homogenous distribution of the nanoparticles within a hydrogel. As another example, MSNs and dMSNs of embodiments may be modified with organotrialkoxysilanes or organotrichlorosilanes to enhance drug-particle interactions.

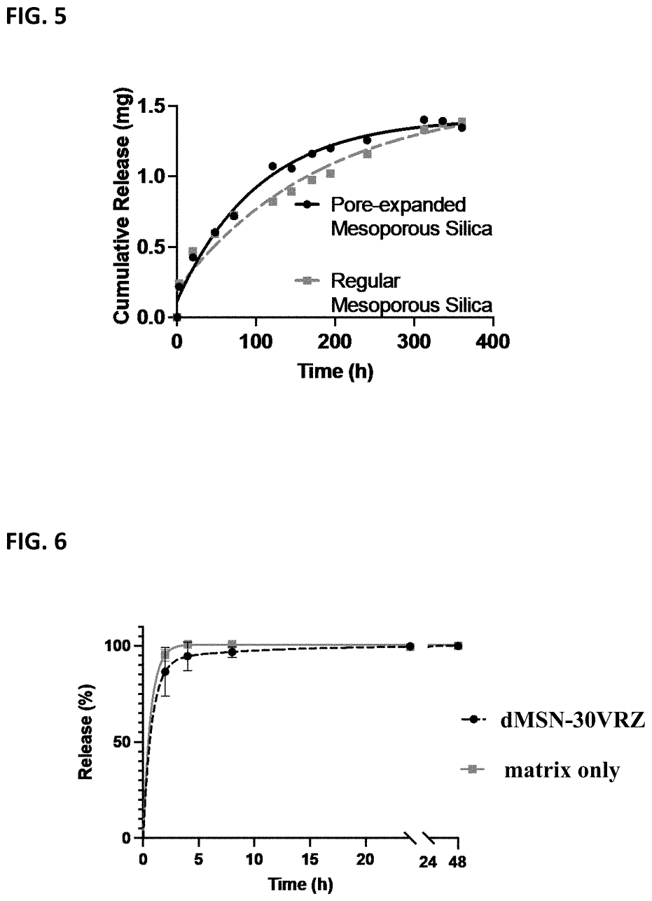

In addition, in some embodiments, the surface properties of the porous nanoparticles, such as pore diameter and specific area, are modified to tune their drug loading capacity and drug release profile. For example, FIG. 5 illustrates the drug release profile from MSNs with different pore diameters according to many embodiments. More specifically, the top plot in FIG. 5 shows the drug release profile for the release of an antifungal drug natamycin into a phosphate-buffered saline solution by pore-expanded mesoporous silica nanoparticles with 4 nm diameter pores over a two-week period, while the bottom plot shows the same for MSNs with “regular” (i.e., non-expanded) 2 nm dimeter pores. In this example, both types of MSNs show similar drug release profiles at the beginning of the two-week observation period; however, MSNs with the smaller pore diameter of 2 nm show slower natamycin release at the later stage. Overall, extended delivery of natamycin from mesoporous silica in solution was achieved for over 10 days from both types of MSNs.

Not to be bound by any theory, it is expected that the release profile of the composite materials, wherein drug-loaded porous nanoparticles are embedded within a polymeric matrix, can be further modified, beyond what has been demonstrated for, for example, free floating MSNs in a liquid medium, because a properly chosen polymeric matrix may serve as a diffusion barrier for the drug contained within the nanoparticles. Furthermore, other advantages of embedding drug loaded nanoparticles into a polymeric matrix according to the instant disclosure may include, but are not limited to: 1) the polymeric matrix serves as a mechanical support for the drug-loaded particles; and 2) the polymeric matrix provides means for fine tuning the drug release profile of the composite material, wherein the matrix composition and or crosslinking density can affect the diffusivity of the composite material and the drug-matrix interaction. Accordingly, in many embodiments, embedding drug loaded nanoparticles into a polymeric matrix allows for easy application and removal of the composite material, and affords the desired drug release profile from the composite material to the tissue being treated upon contact.

To this end, as another example, FIG. 6 illustrates the drug release profile for the composite material of many embodiments and compares it to the drug release profile for a similar sample that comprises the same drug but loaded directly into the polymeric matrix, without the use of any porous nanoparticles as drug carriers. Here, both samples are planar films, with pre-hydration dimensions of 2 cm×2 cm and average thickness of 57 μm, loaded with voriconazole; wherein the composite material sample comprises voriconazole-loaded 60 to 80 nm dMSNs with 6 to 10 nm pores embedded in chitosan matrix (dMSN-30 VRZ); and the directly drug loaded matrix sample only comprises the same chitosan matrix and voriconazole. Furthermore, for the drug-release measurements, both samples (taken dry) were immersed into 5 ml of a phosphate-buffered solution (PBS) at pH 7.4, and the measurements were taken at 34° C. with 100 rpm shaking. As seen from FIG. 6, the voriconazole release by such sample of the composite material lasted for 24 hours, at which point it neared a plateau. Accordingly, in many embodiments, a BCL comprising the composite material with at least such or similar composition and parameters may represent a suitable candidate for applications as a disposable lens for daily treatment and or prophylaxis of an eye condition. In contrast, the sample wherein voriconazole was directly loaded into the polymeric matrix reached the release plateau in just 4 h. Therefore, in many embodiments, the composite materials of the instant application demonstrate an extended drug release profile, as compared to materials that do not employ porous nanoparticles for drug loading.

In many embodiments, the chemical, physical, and or textural surface properties of mesoporous silica or zeolite nanoparticles are adjusted to achieve an optimal drug loading capacity and drug release profile/elution performance according to the desired therapeutic requirements. For example, in many embodiments, optimally maximized specific area of mesoporous silica or zeolite nanoparticles offers greater extent of drug-particle interaction and inhibits crystallization of drugs, which, in turn, increases the drug solubility and bioavailability, wherein low solubility of drugs is one of the main challenges associated with drug delivery methods involving nanoparticles. In many embodiments, the same is achieved via optimization of the pore diameter. In addition, in many embodiments, the highly porous structure of mesoporous silica or zeolite offers higher loading capacity than other types of drug delivering nanoparticles, which also increases the bioavailability of the dugs delivered by the composite materials of the instant application.

In many embodiments, mesoporous silica or zeolite nanoparticles are chosen as drug carrying vehicles for their excellent resistance to adverse processing conditions of various fabrication processes. For example, in many embodiments, the mesoporous silica or zeolite nanoparticles shield and protect the medication molecules they contain from UV radiation. More specifically, the fabrication of commercial contact lenses relies on UV curing, which can cause degradation of the drugs molecules loaded directly into a contact lens. Accordingly, in many embodiments, the MSNs and zeolite nanoparticles of the application provide effective UV-shielding during the contact lens fabrication process and prevent drug molecule degradation caused by UV curing of the lens. In some other embodiments, mesoporous silica or zeolite nanoparticles are the preferred choice over other conventional drug delivery vehicles, such as poly(lactic-co-glycolic acid) (PLGA) nanoparticles or liposomes, because mesoporous silica or zeolite nanoparticles maintain particle integrity in, for example, an acidic processing environment, such as required in the processing of, for example, chitosan, or other biopolymer materials of the polymeric matrix of the embodiments.

In many embodiments, the medication used to impregnate the MSNs or zeolite nanoparticles is a medication selected from the group including, but not limited to: antibiotics, antifungals, anti-inflammatories, antiglaucoma agents, antihistamines, eye lubricants, medications or supplements for dry eye diseases, and other ocular diseases treatable via topical administrations. In some embodiments, the antifungal ophthalmic medication is natamycin or voriconazole. In many embodiments, MSNs or zeolite nanoparticles are loaded with a medication via the solvent evaporation method or absorption. In many such embodiments, drug loading is optimized via adjustment of one of or a combination of: varying initial feed ratios, solvents, and nanoparticle's pore size, nanoparticle's surface functional groups. In many embodiments, the bandage comprising the composite material, and especially the BCL, increases retention time of drugs on the ocular (or another tissue) surface and delivers a therapeutic agent in a sustained manner, hence providing higher bioavailability and efficacy of drug delivery, while also minimizing side effects, such as lack of optical transparency, and enhances patient compliance. In many embodiments, the ophthalmic conditions treated by the BCL include but are not limited to: common ocular diseases including but not limit to dry eye disease, eye allergies, glaucoma, microbial infections, inflammations, and other diseases that can be treated by topical administration. Such contact lenses can also be applied in post-injury or post-surgery treatment to accelerate recovery.

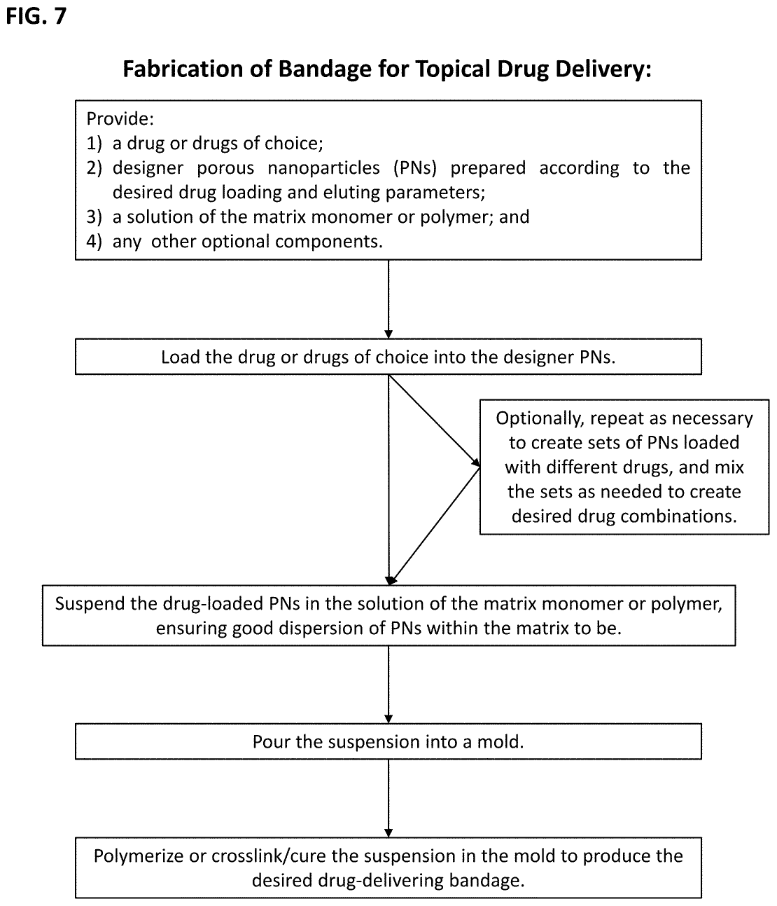

FIG. 7 provides a diagram for one example of the fabrication process to produce a bandage comprising the composite material of the instant disclosure for topical drug delivery according to some embodiments. According to this diagram, in many embodiments, a drug of choice is first loaded into the designer porous nanoparticles (PNs) prepared to accommodate desired therapeutic or prophylactic parameters. In many such embodiments, next, the drug-loaded PNs are suspended in a solution of the matrix monomer or polymer, combined with any other desired or necessary precursors, and thoroughly mixed (e.g., with help of sonication) to ensure good dispersion of PNs within the composite's matrix to be. In many embodiments, the suspension is next poured into a mold for the desired bandage size and shape, such as, for example, a contact lens mold, allowed to set, and polymerized or cured/crosslinked to produce the transparent, flexible, drug-loaded bandage for continuous topical drug application. In many embodiments, the crosslinking method is chosen from the group consisting of: UV-crosslinking, thermal crosslinking, chemical crosslinking and any combination thereof. In many embodiments, wherein the thermal crosslinking is chosen as the crosslinking method, special care is taken to ensure that the crosslinking temperature is below the temperature of degradation for the drug of choice. In many embodiments, the fabrication sequence illustrated by FIG. 7, wherein PNs are pre-loaded with the drug prior to the bandage fabrication, minimizes the time that the drug-loaded nanoparticles spend in solution and, hence, decreases the drug loss during the fabrication process. In addition, the process described in FIG. 7 allows for tunable drug content in the bandage and also for enhanced mechanical strength of the bandage.

In some embodiments, the composite material and, accordingly, the bandage comprising the composite material are fabricated to carry and topically deliver more than one drug. In some such embodiments, the multi-drug loading is achieved by: 1) preparing separate sets of PNs, wherein PNs of each set carry one drug of choice; 2) mixing the PNs pre-loaded with different drugs; and 3) embedding the PN mixture within the polymeric matrix of embodiments. In many embodiments, regardless of the desired number of drugs to be loaded into the bandage, the bandage drug infusion is based on carefully balanced considerations, wherein, for example, optimization of PN drug-carrying capacity (which requires maximizing particle size and concentration) is balanced against the requirement of bandage transparency (which requires minimizing particle size and concentration) to produce the transparent bandage or BCL of embodiments capable of topically delivering the desired amount of each of the at least one drug. In many embodiments, each bandage or BCL is personalized for a single patient's needs, wherein the bandage or BCL is prepared according to the individual needs and or prescription to topically deliver, over the prescribed amount of time, the desired number and amount of drugs to treat a single or multiple medical condition.

In many embodiments, the bandage or BCL comprising the composite material is hydrated and or swelled prior to the intended application. In some embodiments, the bandage is first applied to the intended tissue and only hydrated and or swelled afterwards. In many embodiments, the hydration and or swelling of the composite material is achieved by contacting or immersing the composite material into a solution selected from the group consisting of: water, PBS buffer, artificial tear, saline solution. In many embodiments, the drug-loaded composite materials fabricated according to the processes and procedures described herein, are stored for a period of time prior to use as a bandage or BCL. In many such embodiments, the composite material is stored in a drug saturating buffer solution, such that the drug content and hydration level of the composite material is maintained during the storage. However, in some embodiments, the composite material is stored dry and only hydrated and or swelled immediately prior or after its application to a tissue in need of treatment.

Accordingly, the present disclosure is also directed to a kit for bandaging a tissue in need of topical drug application and protection from the environment. In many embodiments, the kit is useful for facile and secure bandaging of the said tissue with a reliable outcome. In many embodiments, the kit at least comprises some amount of the composite material pre-loaded with a drug or drugs of interest. In some embodiments, the kit comprises the composite material in the form of a dry, bulk or pre-cut/shaped film. In many such embodiments, the kit also comprises an appropriate hydrating and or swelling solution. However, in some embodiment, the kit's composite material is pre-hydrated and or pre-swelled. In many embodiments, the kit also optionally comprises other useful components, such as, for example and not limited to: appropriate adhesives, buffers, pharmaceutically acceptable carriers, applicators, cutters, molds or other shapers, pipetting or measuring tools, and other useful paraphernalia as will be readily recognized by those of skill in the art. In many embodiments, the materials or components assembled in the kit can be provided to the practitioner and or stored in any convenient and suitable ways that preserve their operability and utility. For example; the kit can be stored and provided at room, refrigerated or frozen temperatures, according to the handling requirements of the drug or drugs within the composite material. The components are typically contained in suitable packaging material(s).

In many embodiments, the exact nature of the components in the kit depends on its intended purpose. For example, in some embodiments, wherein the kit is intended for an ocular drug application, the kit contains the composite material pre-shaped into a contact lens and pre-loaded with a drug or drugs for treatment of the intended eye condition. In some embodiments, the kit contains a variety of composite materials, each comprising a different amount of the pre-loaded drug, and or each comprising a different drug or combination of drugs, to accommodate a treatment plan of any duration and complexity.

In many embodiments, the bandage of the instant disclosure comprises a transparent, flexible composite material comprising a plurality of drug-loaded porous silica nanoparticles embedded into a matrix comprising chitosan hydrogel crosslinked by TPP or genipin. In many embodiments, such composite material has the desired drug release profile, while demonstrating excellent microbial inhibition and corneal epithelial cell survival on the surfaces of the matrix. In many embodiments, the bandage is shaped into a lens with superb therapeutic efficacy against many ophthalmic conditions, such as, for example, fungal keratitis.

### EXEMPLARY EMBODIMENTS

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight in g/mol, temperature is in degrees Celsius, and pressure is at or near atmospheric. Standard abbreviations may be used, e.g., s or sec, second(s); min, minute(s); h or hr., hour(s); and the like.

### Example 1—Examplary Synthesis of MSNs

An aqueous solution of cetyltrimethylammonium bromide (CTAB) (2.08 mM, 480 mL) was first mixed with an aqueous solution of sodium hydroxide (NaOH) (2 M, 3.5 mL) and, optionally, mesitylene (7 mL) as a pore expanding agent to a final concentration of 1.4 vol % of mesitylene. Next, tetraethyl orthosilicate (TEOS) was added dropwise to the vigorously stirred solution of CTAB, NaOH, and mesitylene, and the reaction was allowed to continue for another 2 hours. After 2 hours, the reaction precipitate was collected, washed with abundant amount of methanol, and treated to an acid extraction to remove CTAB to produce the desired MSNs of two types (depending on the addition of the pore expanding agent) with the following parameters: 1) 750 nm mean particle diameter and 2.2 nm pore diameter; and 2) 680 nm mean particle diameter and 3.8 to 4.2 nm pore diameter for expanded pore particles, respectively.

### Example 2—Examplary Synthesis of dMSNs

CTAB (1.92 g), triethanolamine (TEA) (35 g), and Capstone FS-66 (0.32 g) were mixed in 100 mL of water, and the pH of the mixture was brought to 7.4 by adding diluted phosphate acid. The solution was then stirred and reacted at 60° C. for 1 h. Next, 15 ml of TEOS was added to the solution and further reacted at the same temperature for another 4 hours. After 4 hours, the obtained nanoparticles were washed with abundant ethanol, and treated with an ammonium nitrate saturated solution to remove CTAB and Capstone FS-66. The resulting particles were found to have overall particle diameters of 60 to 80 nm, with pore diameters of 6 to 10 nm.

### Example 3—Drug-Loading of MSNs and dMSNs

MSNs fabricated according to the protocol described in Example 1 above were loaded with natamycin, a standard medication for fungal keratitis, via either a solvent evaporation method or absorption method. The drug loading efficiencies were investigated by varying initial feed ratio, solvent, and MSN pore size. The particles' drug content was verified by thermogravimetric analysis, while their drug release profile was characterized by UV-Vis spectroscopy. It was found that loading via adsorption equilibrium affords low drug content of ˜3 wt % in MSNs, while loading via solvent evaporation may yield much higher drug content, as high as 20 to 50 wt % in MSNs, depending on the initial drug-particle feed ratio. Therefore, the solvent evaporation method afforded higher drug content in the treated particles than the absorption method. Similar procedures have been employed for loading voriconazole into dMSNs of Example 2 above.

### Example 4—Preparation of the Composite Material from dMSNs and Chitosan

Empty dMSNs with diameters in the range of 60 to 80 nm and 6 to 10 nm pores were provided. An antifungal drug voriconazole was next loaded into the dMSNs via solvent evaporation as described in Example 3 above, such that dMSNs with a 30 wt % of voriconazole load were obtained—dMSN-30VRZ. Next, the desired amount (to afford the composite material with 13 wt % of dMSNs) of dMSN-30VRZ (or empty dMSNs for a control procedure) was suspended in water and sonicated. Chitosan powder was then added to the suspension of dMSN-30VRZ (or empty dMSNs) in water, along with 1M aqueous solution of acetic acid, in the amounts necessary to achieve a final concentration of 2 wt % chitosan in 0.1 M acetic acid. The viscosity of the 2 wt % chitosan in 0.1 M acetic acid was 420 cP. The resulting mixture was stirred for 15 to 30 min to ensure full dissolution of chitosan. Next, the mixture was either drop cast onto a planar mold and left to air dry to produce a flat film, or cast onto a concaved mold and spun at 120 to 150 rpm while drying to produce a lens, both constructs comprising a hydrogel chitosan matrix embedded with the drug-carrying (or “control” empty) dMSNs.

In addition, to neutralize and crosslink the chitosan hydrogel, the dried film and lens, were: 1) immersed in aqueous 0.1 M NaOH solution for 1 min; 2) rinsed with pure water; 3) immersed in 5 wt % or 10 wt % aqueous sodium tripolyphosphate (TPP) solution for 1 hour; and 4) rinsed with pure water again. The resulting film and lens were next tapped dry and left to air dry overnight or until further use.

### DOCTRINE OF EQUIVALENTS

This description of the invention has been presented for the purposes of illustration and description. It is not intended to be exhaustive or to limit the invention to the precise form described, and many modifications and variations are possible in light of the teaching above. The embodiments were chosen and described in order to best explain the principles of the invention and its practical applications. This description will enable others skilled in the art to best utilize and practice the invention in various embodiments and with various modifications as are suited to a particular use. The scope of the invention is defined by the following claims.

1. A bandage contact lens for topical administration of ophthalmic drugs comprising
a composite material comprising:
a matrix comprising a polymeric material;
a plurality of porous nanoparticles embedded within the matrix and characterized by a mean particle diameter and a pore diameter; and
at least one drug for treatment of an ophthalmic condition loaded into the plurality of porous nanoparticles; wherein
the composite material is transparent and flexible, and delivers the at least one drug to an eye topically and consistently upon contact over a period of time, while also protecting the eye from the environment.

2. The bandage contact lens of claim 1, wherein the ophthalmic condition is a medical condition selected from the group consisting of: dry eye disease, eye allergy, glaucoma, microbial infection, inflammation, post-trauma and post-surgery care, any combination thereof, and any other ocular condition that can be treated by topical administrations of a drug.

3. The bandage contact lens of claim 1, wherein the at least one drug is selected from the group consisting of: antibiotics, antifungals, anti-inflammatories, antiglaucoma agents, antihistamines, eye lubricants, medications and supplements for dry eye disease, medication for any other ocular diseases treatable via topical administration, and any combination thereof.

4. The bandage contact lens of claim 3, wherein the at least one drug is selected from the group consisting of: natamycin, voriconazole, and any combination thereof.

5. The bandage contact lens of claim 1, wherein the plurality of porous nanoparticles is a material selected from the group consisting of: mesoporous silica nanoparticles, dendritic mesoporous silica nanoparticles, zeolite nanoparticles, and any combination thereof.

6. The bandage contact lens of claim 1, wherein the mean particle diameter is less than 400 nm.

7. The bandage contact lens of claim 6, wherein the mean particle diameter is less than 200 nm.

8. The bandage contact lens of claim 1, wherein the plurality of porous nanoparticles is chemically modified at the surface.

9. The bandage contact lens of claim 1, wherein the matrix comprises an inherently antimicrobial material.

10. The bandage contact lens of claim 1, wherein the matrix comprises a material selected from the group consisting of: a biopolymer, any commercial polymer used in manufacturing of soft contact lenses, including polyhydroxyethylmethacrylate, polydimethyl siloxane, polydimethylsiloxane copolymers, and any combination thereof.

11. The bandage contact lens of claim 10, wherein the biopolymer is a polymer selected from the group consisting of: chitosan, chitosan crosslinked with genipin or tripolyphosphate, silk Fibron, gelatin, any combination thereof.

12. The bandage contact lens of claim 1, wherein the period of time is at least 8 hours.

13. The bandage contact lens of claim 1, wherein the plurality of porous nanoparticles is dendritic mesoporous silica nanoparticles, the at least one drug is natamycin, and the matrix comprises chitosan crosslinked with tripolyphosphate.

14. A bandage for topical drug delivery comprising
a composite material comprising:
a matrix comprising a polymeric material;
a plurality of porous nanoparticles embedded within the matrix, and characterized by a mean particle diameter and a pore diameter; and
at least one drug for treatment of a medical condition loaded into the plurality of porous nanoparticles; wherein
the composite material is transparent, flexible, and delivers the at least one drug to a tissue topically and consistently upon contact over a period of time, while also protecting the tissue from the environment.

15. The bandage of claim 14, wherein the at least one drug is selected from the group consisting of: antibiotics, antifungals, anti-inflammatories, antihistamines, lubricants, medication for any disease or condition treatable via topical administration, and any combination thereof.

16. The bandage of claim 14, wherein the plurality of porous nanoparticles is a material selected from the group consisting of: mesoporous silica nanoparticles, dendritic mesoporous silica nanoparticles, zeolite nanoparticles, and any combination thereof.

17. The bandage of claim 14, wherein the plurality of porous nanoparticles is chemically modified at the surface.

18. The bandage of claim 14, wherein the matrix comprises an inherently antimicrobial material.

19. The bandage of claim 14, wherein the matrix comprises a material selected from the group consisting of: a biopolymer, poly(hydroxyalkylmethacrylate), poly(alkylmethacrylate), poly(bisphenol-A carbonate), poly(alkylsiloxane), copolymers of poly(alkylsiloxane), and any combination thereof.

20. The bandage of claim 14, wherein the biopolymer is a polymer selected from the group consisting of: chitosan, chitosan crosslinked with genipin or tripolyphosphate, silk Fibron, gelatin, and any combination thereof.

21. The bandage of claim 14, wherein the period of time is at least 8 hours.

22. A method of sustained topical administration of an ophthalmic drug comprising:
providing a bandage contact lens for topical of ophthalmic drugs comprising
a composite material comprising:
a matrix comprising a polymeric material;
a plurality of porous nanoparticles embedded within the matrix, and characterized by a mean particle diameter and a pore diameter; and
at least one drug for treatment of an ophthalmic condition loaded into the plurality of porous nanoparticles; wherein
the composite material is transparent and delivers the at least one drug to an eye topically and consistently upon contact over a period of time, while also protecting the eye from the environment; and
applying the bandage contact lens to the eye to treat the ophthalmic condition.

23. A method of sustained topical administration of a medicament comprising:
providing a bandage for topical drug delivery comprising
a composite material comprising:
a matrix comprising a polymeric material;
a plurality of porous nanoparticles embedded within the matrix, and characterized by a mean particle diameter and a pore diameter;
at least one drug for treatment of a medical condition loaded into the plurality of porous nanoparticles; wherein
the composite material is transparent and delivers the at least one drug to a tissue topically and consistently upon contact over a period of time, while also protecting the tissue from the environment; and
applying the bandage to the tissue to treat the medical condition.

24. A composite material comprising:
a matrix comprising a polymeric material;
a plurality of porous nanoparticles embedded within the matrix, and characterized by a mean particle diameter and a pore diameter; and
at least one drug for treatment of a medical condition loaded into the plurality of porous nanoparticles; wherein
the composite material is transparent and flexible, and delivers the at least one drug to a tissue topically and consistently upon contact over a period of time, while also protecting the tissue from the environment.

25. The composite material of claim 24, wherein the plurality of porous nanoparticles is a material selected from the group consisting of: mesoporous silica nanoparticles, dendritic mesoporous silica nanoparticles, zeolite nanoparticles, and any combination thereof.

26. The composite material of claim 24, wherein the matrix comprises a material selected from the group consisting of: chitosan, chitosan crosslinked with genipin or tripolyphosphate, silk Fibron, gelatin, and any combination thereof.
